(19) United States
(12) Patent Application Publication
(10) Pub. No.: US 2026/0250682 A1
(43) Pub. Date: Aug. 27, 2026

(54) miRNA PC-5P-12969 Based Therapeutic Approach for Alzheimer's Disease

(71) Applicant: Texas Tech University System, Lubbock, TX (US)

(72) Inventors: P. Hemachandra Reddy, Lubbock, TX (US); Murali Vijayan, Lubbock, TX (US)

(73) Assignee: Texas Tech University System, Lubbock, TX (US)

(21) Appl. No.: 19/550,564

(22) Filed: Feb. 26, 2026

Related U.S. Application Data
(60) Provisional application No. 63/763,781, filed on Feb. 26, 2025.

(51) Int. Cl. C12N 15/113; A61K 31/7105; A61K 38/00; C12Q 1/6883

(52) U.S. Cl. CPC C12N 15/113; A61K 31/7105; A61K 38/00; C12Q 1/6883; C12Q 2600/118

(57) ABSTRACT

The invention relates to methods of treating or preventing a neurological disorder in a subject by administering to the subject at least one microRNA (miRNA), at least one inhibitor of the miRNA, or combinations thereof. The miRNA to be administered or inhibited includes, without limitation, miR PC-5P-12969 (SEQ ID NO: 1), an analog thereof, a derivative thereof, or combinations thereof. The invention also relates to compositions that include the at least one microRNA (miRNA), the at least one inhibitor of the miRNA, or combinations thereof. The invention further relates to methods of diagnosing a neurological disorder in a subject by (1) obtaining a biological sample from the subject; (2) detecting miR PC-5P-12969 (SEQ ID NO: 1) levels in the biological sample; and (3) correlating overexpressed levels of miR PC-5P-12969 in the biological sample to the presence of the neurological disorder in the subject.

### CROSS-REFERENCE TO RELATED APPLICATIONS

This application claims priority to U.S. Provisional Patent Application No. 63/763,781 filed on Feb. 26, 2025. The entirety of the aforementioned application is incorporated herein by reference.

### SEQUENCE DISCLOSURE STATEMENT

Pursuant to 37 C.F.R. § 1.834, Applicant has submitted a sequence listing in XML format (“Sequence Listing”). The name of the file containing the Sequence Listing is “AF13368.P081US.xml”. The date of the creation of the Sequence Listing is Feb. 26, 2026. The size of the Sequence Listing is 14,000 bytes. Applicant hereby incorporates by reference the material in the Sequence Listing.

### BACKGROUND

A need exists for more effective compositions and methods for treating and diagnosing various neurological disorders. Numerous embodiments of the present disclosure aim to address the aforementioned need.

### SUMMARY

In some embodiments, the present disclosure pertains to methods of treating or preventing a neurological disorder in a subject. In some embodiments, the methods of the present disclosure include administering to the subject at least one microRNA (miRNA), at least one inhibitor of the miRNA, or combinations thereof. In some embodiments, the miRNA to be administered or inhibited includes, without limitation, miR PC-5P-12969 (SEQ ID NO: 1), an analog thereof, a derivative thereof, or combinations thereof.

Additional embodiments of the present disclosure pertain to compositions that include the at least one microRNA (miRNA), an expression vector expressing the at least one miRNA, at least one inhibitor of the miRNA, or combinations thereof. In some embodiments, the composition is suitable for use in treating or preventing a neurological disorder in a subject.

Further embodiments of the present disclosure pertain to methods of diagnosing a neurological disorder in a subject. In some embodiments, such methods include: (1) obtaining a biological sample from the subject; (2) detecting miR PC-5P-12969 (SEQ ID NO: 1) levels in the biological sample; and (3) correlating overexpressed levels of miR PC-5P-12969 in the biological sample to the presence of the neurological disorder in the subject.

### BRIEF DESCRIPTION OF THE DRAWINGS

FIGS. 1A-1C provide data regarding the expression of miRNA PC-5P-12969. As shown in FIG. 1A, upregulated miRNA PC-5P-12969 expression levels were examined in postmortem brain tissues from individuals with Alzheimer's disease (AD) (n=27) and healthy control brain tissues (n=15). Significant difference among groups were calculated by unpaired T-test, where p<0.05 is considered statistically significant. FIG. 1B shows upregulated miRNA PC-5P-12969 expression levels in different AD transgenic mice. Expression levels of miRNA PC-5P-12969 were measured in tissue samples of AβPP (6 months), TAU (6 months), hAβ-KI (6 and 12 months) mice (n=5; five different animals were used from each age group selected for the study). FIG. 1C shows quantitative measurement of miRNA PC-5P-12969-fold change expression after 24-48 h of the miRNA PC-5P-12969 vector and the mAPP cDNA transfecting cells. Data are from three independent experiments with similar results (n=3). Significant difference among groups were calculated by one-way ANOVA with p<0.05 is considered statistically significant.

FIGS. 2A-2B show the effects of miRNA PC-5P-12969 and mutant APP vectors on cell viability and apoptotic cell death. FIG. 2A shows that cell apoptosis was significantly increased in mAPP overexpressing-HT22 cells relative to control HT22 cells. Both mAPP and miRNA PC-5P-12969 (Post-condition) led to a significant reduction in apoptotic cell death as compared to control cells. FIG. 2B shows that cell viability was significantly increased in miRNA PC-5P-12969 treated cells than mAPP treated cells. Likewise, mAPP and miRNA PC-5P-12969 vector treated group showed increased cell viability compared to mAPP. Data are from three independent experiments with similar results (n=3). Significant difference among groups were calculated by one-way ANOVA, where p<0.05 is considered statistically significant.

FIGS. 3A-3C show the interaction of miRNA PC-5P-12969 with the GSK3 gene. FIG. 3A shows putative binding sites of miRNA PC-5P-12969 at the 3′-UTR regions of a WT GSK3α gene in human (binding sites are located at sequence positions 2984-3004 and 3434-3456). FIG. 3B shows putative binding sites of miRNA PC-5P-12969 at the 3′-UTR regions of a WT GSK3α gene in mouse (binding site is located at sequence position 1716-1736). FIG. 3C shows normalized luciferase activity (Firefly/Renilla) in HT22 cells co-transfected with the GSK3α 3′-UTR and miRNA PC-5P-12969 agomirs. Applicant examined the firefly/Renilla luciferase activity of the wild-type (WT) GSK3α 3′-UTR, which showed a significant decrease upon treatment with the miRNA PC-5P-12969 agomir. Data are from three independent experiments with similar results (n=3). Significant difference among groups were calculated by one-way ANOVA, where p<0.05 is considered statistically significant.

FIGS. 4A-4D show the regulation of GSK3α and APP gene and protein expression by miRNA PC-5P-12969. FIG. 4A shows a quantitative measurement of GSK3 mRNA folds change expression after 24-48 h of the miRNA PC-5P-12969 vector and the mAPP cDNA transfecting cells. FIG. 4B shows quantitative measurement of APP mRNA folds change expression after 24-48 h of the miRNA PC-5P-12969 vector and the mutant APP cDNA transfecting cells. FIG. 4C shows a western blot for GSK3α and APP (6E10), and B-actin proteins in cells transfected with the miRNA PC-5P-12969 vector, mAPP cDNA and co-transfected cells. FIG. 4D shows quantitative densitometry analysis of GSK3α and AβPP proteins. Data are from three independent experiments with similar results (n=3). Housing-keeping protein beta-actin was used as the loading control. Significant difference among groups were calculated by one-way ANOVA, where p<0.05 is considered statistically significant. *p≤0.05, **p≤0.01, ***p≤0.001, ****p≤0.0001 compared to the control.

FIGS. 5A-5C show the analysis of mitochondrial bioenergetics. FIG. 5A shows that the maximal oxygen consumption rate (OCR) was assessed in mouse hippocampal HT22 cells treated with over expression vector (OEV) of miRNA PC-5P-12969, mAPP, OEV miRNA PC-5P-12969+mAPP (pre-condition), mAPP+ miRNA-PC-5P-12969 (post-condition) and their respective controls alone and/or in combination using an XFe96-well Extracellular Flux Analyzer (Agilent Technologies). FIG. 5B shows that significantly decreased maximal OCR and ATP were observed in HT22 cells treated with mAPP. FIG. 5C shows that, at the same time, increased maximal OCR was observed in OEV miRNA PC-5P-12969 and mAPP+ miRNA PC-5P-12969 (post-condition) groups. The results indicate that mAPP decreases the maximal OCR and ATP production and overexpression of miRNA PC-5P-12969 rescues the OCR in the mutant APP condition. Data are from three replicates from three independent experiments with similar results (n=3). Results were expressed as mean±SEM, one-way ANOVA followed by Turkey's test for multiple comparisons.

FIGS. 6A-6C show the analysis of mitochondrial structure and length. FIG. 6A shows representative TEM images of HT22 cells transfected with the miRNA PC-5P-12969 vector, mAPP cDNA, co-transfected with mAPP and miRNA PC-5P-12969, showing mitochondrial organization. FIG. 6B represents the mitochondrial number in the HT22 cells transfected with the miRNA PC-5P-12969 vector, mAPP cDNA, co-transfected with mAPP and miRNA PC-5P-12969. FIG. 6C represents the mitochondrial length in the HT22 cells transfected with the miRNA PC-5P-12969 vector, mAPP cDNA, co-transfected with mAPP and miRNA PC-5P-12969. Results were expressed as mean±SEM, one-way ANOVA followed by Turkey's test for multiple comparisons (scale bar=500 nm).

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Current compositions and methods of treating various neurological disorders, such as Alzheimer's Disease (AD), have numerous limitations. For instance, the intricate and complex molecular mechanisms that underlie the progression of AD have prompted a concerted and vigorous research endeavor aimed at uncovering potential avenues for therapeutic intervention.

In fact, AD stands as one of the most intricate and devastating neurodegenerative disorders that affect the elderly. Moreover, as the elderly population continues to grow, the global burden of AD intensifies, thereby presenting substantial hurdles for healthcare systems worldwide.

Despite extensive research spanning decades and notable strides in comprehending the molecular basis of AD, the quest for effective therapies capable of halting or reversing its advancement remains elusive. Throughout recent decades, AD has emerged as a prevalent and incapacitating age-related ailment. The World Health Organization (WHO) estimates that about 60 million individuals currently grapple with dementia, and AD accounts for roughly 60-70% of dementia cases.

However, the pursuit of viable treatments for AD and other neurological disorders has encountered formidable obstacles, with numerous pharmaceutical trials encountering setbacks and frustrations. While specific medications for AD and other neurological disorders have gained approval for symptom management and the deceleration of cognitive decline, they fail to furnish a cure or put a halt to the fundamental progression of the disease.

As such, a need exists for more effective compositions and methods for treating various neurological disorders, such as AD. Numerous embodiments of the present disclosure aim to address the aforementioned need.

In some embodiments, the present disclosure pertains to methods of treating or preventing a neurological disorder in a subject. In some embodiments, the methods of the present disclosure include administering to the subject at least one microRNA (miRNA), at least one inhibitor of the miRNA, or combinations thereof. In some embodiments, the miRNA to be administered or inhibited includes, without limitation, miR PC-5P-12969 (SEQ ID NO: 1; DNA Sequence: GCAGGAGCCGGGACTGGCTTC; RNA Sequence: GCAGGAGCCGGGACUGGCUUC), an analog thereof, a derivative thereof, or combinations thereof.

Additional embodiments of the present disclosure pertain to compositions that include the at least one microRNA (miRNA), an expression vector expressing the at least one miRNA, at least one inhibitor of the miRNA, or combinations thereof. In some embodiments, the composition is suitable for use in treating or preventing a neurological disorder in a subject.

Further embodiments of the present disclosure pertain to methods of diagnosing a neurological disorder in a subject. In some embodiments, such methods include: (1) obtaining a biological sample from the subject; (2) detecting miR PC-5P-12969 (SEQ ID NO: 1) levels in the biological sample; and (3) correlating overexpressed levels of miR PC-5P-12969 in the biological sample to the presence of the neurological disorder in the subject. In some embodiments, the diagnostic methods of the present disclosure also include a step of (4) implementing a treatment decision based on the diagnosis. In some embodiments, the treatment decision includes administering a therapeutic agent to the subject. In some embodiments, the therapeutic agent includes an inhibitor of miR PC-5P-12969.

As set forth in more detail herein, the methods and compositions of the present disclosure can have numerous embodiments.

miRNAs

The methods of the present disclosure may be used to administer or inhibit various miRNAs in subjects. Additionally, the compositions of the present disclosure can include various miRNAs. Moreover, the diagnostic methods of the present disclosure may be utilized to detect various miRNAs from biological samples. For instance, in some embodiments, the miRNA includes, without limitation, miR PC-5P-12969 (SEQ ID NO: 1), an analog thereof, a derivative thereof, or combinations thereof.

In some embodiments, the miRNA includes a derivative of miR PC-5P-12969. In some embodiments, the miRNA derivative shares at least 70% sequence identity with miR PC-5P-12969. In some embodiments, the miRNA derivative shares at least 75% sequence identity with miR PC-5P-12969. In some embodiments, the miRNA derivative shares at least 80% sequence identity with miR PC-5P-12969. In some embodiments, the miRNA derivative shares at least 85% sequence identity with miR PC-5P-12969. In some embodiments, the miRNA derivative shares at least 90% sequence identity with miR PC-5P-12969. In some embodiments, the miRNA derivative shares at least 95% sequence identity with miR PC-5P-12969. In some embodiments, the miRNA derivative shares at least 99% sequence identity with miR PC-5P-12969.

The miRNAs of the present disclosure can be in various forms. For instance, in some embodiments, the miRNAs of the present disclosure may be part of an expression vector that expresses the miRNA sequence. In some embodiments, the expression vector can include a plasmid. In some embodiments, the miRNAs of the present disclosure may be in isolated form. In some embodiments, the miRNAs of the present disclosure may be in the form of an miRNA precursor that is processed to form an miRNA.

miRNA Inhibitors

The miRNA inhibitors of the present disclosure generally refer to molecules or sequences that can reduce or inhibit the activity of at least one miRNA of the present disclosure (e.g., a miRNA of SEQ ID NO: 1). The diagnostic, treatment and prevention methods of the present disclosure may be used to administer various miRNA inhibitors to subjects. Additionally, the compositions of the present disclosure can include various miRNA inhibitors.

For instance, in some embodiments, the miRNA inhibitor includes a reverse complement sequence of the miRNA to be inhibited. In some embodiments, the miRNA inhibitor includes a reverse complement sequence of SEQ ID NO: 1.

The miRNA inhibitors of the present disclosure can be in various forms. For instance, in some embodiments, the miRNA inhibitors of the present disclosure are in forms that include, without limitation, antisense miRNA oligonucleotides (AMOs), antagomirs, locked nucleic acid (LNA)-modified oligonucleotides, peptide nucleic acid (PNA)-based inhibitors, morpholino antisense oligonucleotides, chemically modified antisense oligonucleotides, or combinations thereof.

Administration of the miRNAs and/or miRNA Inhibitors

The miRNAs and miRNA inhibitors of the present disclosure may be administered to subjects in various manners. For instance, in some embodiments, administration occurs by methods that include, without limitation, intravenous administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, or combinations thereof. In some embodiments, the administration occurs by intracranial administration.

In some embodiments, the methods of the present disclosure include a step of administering at least one inhibitor of a miRNA of the present disclosure. In some embodiments, the methods of the present disclosure include a step of administering at least one miRNA. In some of such embodiments, the administration includes administering an expression vector that expresses the miRNA. In some embodiments, the administration includes administering an miRNA in isolated form.

### Neurological Disorders

The methods of the present disclosure may be utilized to treat or prevent various neurological disorders. For instance, in some embodiments, the neurological disorder includes, without limitation, Alzheimer's Disease (AD), Huntington's Disease, Parkinson's Disease, Amyotrophic Lateral Sclerosis (ALS), Multiple Sclerosis (MS), dementia, mild cognitive impairment (MCI), Schizophrenia, or combinations thereof. In some embodiments, the neurological disorder to be treated or prevented includes Alzheimer's Disease (AD).

The methods of the present disclosure can have various effects on a neurological disorder. For instance, in some embodiments, the methods of the present disclosure prevent a neurological disorder. In some embodiments, the methods of the present disclosure treat a neurological disorder. In some embodiments, the methods of the present disclosure treat and prevent a neurological disorder.

### Subjects

The methods of the present disclosure may be utilized to treat or prevent neurological disorders in various subjects. Additionally, the diagnostic methods of the present disclosure may obtain biological samples from various subjects. For instance, in some embodiments, the subject is a human being. In some embodiments, the subject is a non-human animal. In some embodiments, the non-human animal is a livestock, a cattle, a swine, a poultry, a goat, a companion animal, a dog, a cat, a horse, a rodent, or a primate. In some embodiments, the subject is suffering from a neurological disorder. In some embodiments, the subject is vulnerable to a neurological disorder. In some embodiments, the subject exhibits symptoms of a neurological disorder.

### Biological Samples

The diagnostic methods of the present disclosure may obtain various biological samples from subjects. For instance, in some embodiments, the biological sample includes a brain tissue of a subject. In some embodiments, the brain tissue is obtained from the frontal cortex, the hippocampus, the entorhinal cortex, or combinations thereof. In some embodiments, the brain tissue is obtained from the frontal cortex. In some embodiments, the biological sample includes, without limitation, a blood sample (e.g., a serum sample, a plasma sample, or a whole blood sample), peripheral nerve tissues, cerebrospinal fluids, or combinations thereof.

Detection of miRNA from Biological Samples

The diagnostic methods of the present disclosure may detect miRNA from biological samples in various manners. For instance, in some embodiments, detection occurs by a method that includes, without limitation, a polymerase chain reaction (PCR), a reverse transcriptase (RT) PCR, digital droplet PCR, microarray-based miRNA profiling, next-generation sequencing (NGS), northern blotting, in situ hybridization, or combinations thereof. In some embodiments, detection occurs by RT-PCR.

### Compositions

The compositions of the present disclosure can include various concentrations of miRNAs and miRNA inhibitors. For instance, in some embodiments, the compositions of the present disclosure include miRNA concentrations of at least 1 wt %. In some embodiments, the compositions of the present disclosure include miRNA concentrations of at least 5 wt %. In some embodiments, the compositions of the present disclosure include miRNA concentrations of at least 10 wt %. In some embodiments, the compositions of the present disclosure include miRNA inhibitor concentrations of at least 1 wt %. In some embodiments, the compositions of the present disclosure include miRNA inhibitor concentrations of at least 5 wt %. In some embodiments, the compositions of the present disclosure include miRNA inhibitor concentrations of at least 10 wt %.

In some embodiments, the compositions of the present disclosure include an expression vector that expresses the miRNA. In some embodiments, the expression vector can include a plasmid.

The compositions of the present disclosure can include additional components. For instance, in some embodiments, the compositions of the present disclosure also include at least one pharmaceutically acceptable carrier. In some embodiments, the pharmaceutically acceptable carrier includes at least one excipient. In some embodiments, the excipient includes, without limitation, anti-adherents, binders, coatings, colors, disintegrants, flavors, glidants, lubricants, preservatives, sorbents, sweeteners, vehicles, or combinations thereof.

In some embodiments, the compositions of the present disclosure also include at least one solubilizing agent. In some embodiments, the solubilizing agent includes, without limitation, polyethylene glycol, glycerin, propylene glycol, ethanol, sorbitol, polyoxyethylated glycerides, polyoxyethylated oleic glycerides, polysorbates, sorbitan monooleate, hydroxypropyl-beta-cyclodextrin (HPCD), polyoxyl 40 hydrogenated castor oil, polyoxyl hydroxystearates, or combinations thereof.

The compositions of the present disclosure may be in various forms. For instance, in some embodiments, the compositions of the present disclosure may be associated with a delivery agent. In some embodiments, the delivery agent includes, without limitation, nanoparticles, lipid nanoparticles (LNPs), viral vectors (e.g., adeno-associated viruses and lentiviruses), exosomes, or combinations thereof.

### ADDITIONAL EMBODIMENTS

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Unveiling the Role of Novel miRNA PC-5P-12969 in Alleviating Alzheimer's Disease and Other Neurological Disorders

The intricate and complex molecular mechanisms that underlie the progression of Alzheimer's disease (AD) have prompted a concerted and vigorous research endeavor aimed at uncovering potential avenues for therapeutic intervention. This Example aims to elucidate the role of miRNA PC-5P-12969 (SEQ ID NO: 1) in the pathogenesis of AD.

Applicant assessed the differential expression of miRNA PC-5P-12969 in postmortem AD brains, AD animal and cell models using real-time reverse-transcriptase (RT)-PCR. Applicant also checked the gene and protein expression of GSK3α and APP.

Applicant's investigation revealed a notable upregulation of miRNA PC-5P-12969 in postmortem brains of AD patients, in transgenic mouse models of AD, and in mutant APP overexpressing-HT22 cells. Additionally, Applicant's findings indicate that overexpression of miRNA PC-5P-12969 exerts a protective effect on cell survival, while concurrently mitigating apoptotic cell death. Furthermore, Applicant established a robust and specific interaction between miRNA PC-5P-12969 and GSK3α. Applicant's luciferase reporter assays provided confirmation of the binding between miRNA PC-5P-12969 and the 3′-UTR of the GSK3α gene. Manipulation of miRNA PC-5P-12969 levels in cellular models of AD yielded noteworthy alterations in the gene and protein expression levels of both GSK3α and APP.

Remarkably, the manipulation of miRNA PC-5P-12969 levels yielded significant enhancements in mitochondrial respiration and ATP production, concurrently with a reduction in mitochondrial fragmentation, thus unveiling a potential regulatory role of miRNA PC-5P-12969 in these vital cellular processes. In summary, this Example sheds light on the crucial role of miRNA PC-5P-12969 and its direct interaction with GSK3α in the context of AD.

### Example 1.1. Background

The neuropathological features characterizing AD include the accumulation of extracellular amyloid-β (Aβ) plaques and intracellular neurofibrillary tangles, which have been extensively investigated for numerous years. Aβ stems from the amyloid-β protein precursor (APP), originating from sequential cleavage by β- and γ-secretases. AβPP plays a pivotal role in multiple physiological functions, including neuronal development, synaptogenesis, and the maintenance of synaptic plasticity. Comprehending the intricate connection between AβPP and the evolution of AD pathology stands as a cornerstone for formulating efficacious treatments and interventions to counteract this debilitating ailment.

GSK3, or glycogen synthase kinase 3, includes two distinct isoforms: GSK3α and GSK3β. Although these isoforms share similar functions, they are encoded by separate genes and possess unique roles. The key molecular mechanisms of GSK3α in AD include tau phosphorylation, neurofibrillary tangle formation, synaptic dysfunction, neuronal apoptosis, and inflammatory responses. GSK3α primarily regulates multiple signaling pathways responsible for controlling cell survival, proliferation, and differentiation. Notably, GSK3α plays a pivotal role in the intricate regulation of the Wnt/β-catenin pathway, which is vital for neuronal development and function. In addition to its involvement in the Wnt/β-catenin pathway, GSK3α also influences the processing of AβPP, leading to increased production of Aβ.

Furthermore, GSK3α has undergone extensive research due to its association with the hyperphosphorylation of tau protein. The interplay between GSK3α, tau, and Aβ suggests a multifaceted role for this kinase in the molecular mechanisms underlying AD.

Research efforts have focused on identifying pharmacological agents that can modulate GSK3α activity as a potential therapeutic strategy for AD. Inhibiting GSK3α activity holds promise in reducing Aβ production, preventing tau hyperphosphorylation, and mitigating neuroinflammation. These potential effects offer hope for the development of disease-modifying treatments for AD.

microRNAs (miRNAs) possess remarkable ability to regulate the expression of specific genes, profoundly impacting developmental processes, cellular balance, and the progression of diseases. As Applicant's comprehension of miRNAs deepens, their involvement in a multitude of cellular activities, including differentiation, proliferation, apoptosis, and immunity, has become increasingly apparent. The dysregulation of miRNA expression has been identified as a factor in several neurodegenerative conditions, including AD, thereby adding a layer of intricacy to the underlying pathology.

Amidst the complex contributors to the development of AD, miRNAs have arisen as crucial participants in governing gene expression and the progression of neurodegenerative mechanisms. Employing miRNAs in therapeutic treatments, while simultaneously mitigating off-target effects, presents a formidable challenge. This difficulty arises from the pervasive regulatory influence of miRNAs.

Nonetheless, the significance of identifying miRNAs as prospective targets extends beyond therapeutic application, encompassing diagnostic and prognostic utility. Even in the face of the complexities posed by miRNA-based therapies, comprehending the roles of miRNAs in disease pathogenesis holds substantial value. It facilitates early disease detection, aids in disease categorization, and fosters the exploration of alternative treatment modalities. Furthermore, this knowledge grants valuable insights into the underlying molecular mechanisms of diseases, offering the potential for the unearthing of innovative therapeutic targets and pathways.

Recent insights indicate that multiple miRNAs possess the capability to target APP mRNA, thereby exerting an influence on its expression levels. Apart from their direct role in targeting APP mRNA, miRNAs also contribute to the production of Aβ through alternative mechanisms. They hold the ability to modulate key enzymes integral to the processing of AβPP, such as β secretase (BACE1) as well as the components of γ-secretase (PSEN1 and PSEN2). Moreover, variations in miRNA expression can impact other pathways linked to AD, encompassing tau hyperphosphorylation, neuroinflammation, and synaptic dysfunction.

These significant findings underscore the pivotal role of miRNAs in fine-tuning AβPP levels, thereby intricately affecting the generation of Aβ and the progression of AD. The potential impact of miRNAs on both AβPP and Aβ production suggests a promising avenue for potential therapeutic interventions in AD.

Strategic approaches aimed at inhibiting miRNAs that promote AβPP expression or enhancing those that suppress it hold the potential to restore equilibrium in AβPP levels, consequently ameliorating the burden of Aβ in the brains of individuals with AD. However, it is important to approach the development of miRNA-based therapies with careful consideration, as miRNAs exert regulatory control over a multitude of genes, thereby raising the possibility of unintended off-target effects.

In AD, disruptions in mitochondrial dynamics, morphology, and function have been identified, resulting in reduced ATP production, heightened oxidative stress, and perturbed calcium homeostasis. These alterations extend down to the ultrastructural level, encompassing changes in mitochondrial size, shape, and distribution, which can directly affect the overall cellular well-being.

The interaction between miRNAs and mitochondrial respiration emerges as a fresh perspective to better grasp the complex mechanisms that underlie the development of AD. miRNAs have exhibited a remarkable ability to intricately govern crucial components of the mitochondrial respiratory chain and its associated processes. Of significant note, irregularities in miRNA expression have been observed in the brains of individuals with AD, hinting at their potential involvement in driving mitochondrial dysfunction.

A comprehensive understanding of the precise interactions between miRNAs and mitochondria, along with their repercussions on cellular energy processes, holds the promise of revealing novel targets for therapeutic intervention. These targets could potentially alleviate mitochondrial deficits and, by extension, mitigate the neurodegenerative effects associated with AD.

MiRNA PC-5P-12969 was originally discovered through analysis of ischemic stroke samples in Applicant's miRNA sequencing dataset. Hum Mol Genet, 2018, 27, 2318-2329. miRNA PC-5P-12969 is the mature miRNA, which consists of 21 nucleotides (SEQ ID NO: 1; GCAGGAGCCGGGACTG-GCTTC), and exhibits expression in both human and mouse models. MiRNA PC-5P-12969 is located on chromosome 2 with the genome ID NC 000068.8, and it resides within exon 2 of the mouse Slco4a1 gene. In humans, miRNA PC-5P-12969 is located on chromosome 9 with Sequence ID: NC 000009.12, and it resides within intron 2 of the human Notch1 gene. Functionally, miRNA PC-5P-12969 appears to be upregulated and associated with crucial roles, including neuron maturation, regulation of mitophagy, histone H3-K9 demethylation, and the negative modulation of NIK/NF-kappaB signaling.

In relation to AD, miRNA PC-5P-12969 has been implicated in the targeting of potential AD-associated genes like APP, BACE1, and GSK3α. However, a comprehensive understanding of the precise molecular interactions between miRNA PC-5P-12969 and the APP, BACE1, and GSK3α genes remains elusive. Specifically, it remains uncertain whether the upregulation of miRNA PC-5P-12969 exerts a protective or detrimental influence on the progression and pathogenesis of AD. Additionally, the impact of miRNA PC-5P-12969 on the processing of AβPP and the subsequent intra- and extracellular production of Aβ through the GSK3α mechanism within AD neurons is not fully clarified.

To address these pivotal inquiries, Applicant addresses the following aspects in the present Example: 1) the identification of heightened expression of miRNA PC-5P-12969 in postmortem AD brain samples, transgenic mouse models of AD, and cell lines simulating AD conditions; 2) the role of miRNA PC-5P-12969 as a regulator of GSK3α; 3) the capacity of miRNA PC-5P-12969 to alleviate toxicity resulting from mAPP (mutant APP); and 4) the impact of miRNA-PC-5P-12969 overexpression on the gene and protein expression of both GSK3α and AβPP.

This investigation delves into the complex interplay between miRNA PC-5P-12969 and AD, shedding light on emerging evidence that underscores miRNA PC-5P-12969's involvement in the pathogenesis of AD. Applicant explores the intricate connections between miRNA PC-5P-12969 regulation, changes in mitochondrial ultrastructure, and alterations in mitochondrial respiration within the AD context. By delving into the regulatory mechanisms and unraveling the functional consequences of the elevated expression of miRNA PC-5P-12969 in the context of AD, Applicant's objective is to provide a comprehensive overview of its significance as a potential therapeutic target.

### Example 1.2. HT22 Cell Culture and Differentiation

To verify the distinct expression pattern of the novel miRNA PC-5P-12969 in an in vitro setting, Applicant conducted experiments using HT22 cells, an immortalized line of mouse hippocampal neurons. These cells were cultured in Dulbecco's Modified Eagle Medium (DMEM, Gibco, catalog no. 11995), supplemented with 10% fetal bovine serum (FBS), as well as penicillin (100 U/ml) and streptomycin (100 g/ml) for antibiotic coverage. The cells were meticulously maintained under tightly controlled conditions, including a constant temperature of 37° C., an air composition of 95%, and a CO₂ concentration of 5%.

Additional essential components for cell culture, such as Minimum Essential Medium (MEM), penicillin/streptomycin, fetal bovine serum, and Trypsin-EDTA, were sourced from GIBCO (Gaithersburg, MD, USA). Prior to their utilization, the HT22 cells were subjected to differentiation in NeuroBasal medium (Invitrogen, Carlsbad, CA), supplemented with 1×N2, for a duration of 24 to 48 h.

### Example 1.3. Postmortem Brains from AD Patients and Controls

The present Example analyzed 42 freshly frozen post-mortem brain samples, specifically from the frontal cortex. This investigation primarily concentrated on 27 cases of AD and 15 control subjects. Among these cases, there were 14 males and 13 females, with ages ranging from 65 to 95 years (mean age: 79.85 years). A total of 15 control subjects, consisting of 8 males and 7 females, with ages ranging from 65 to 103 years (mean age: 79.26 years), were obtained. These brain banks were responsible for obtaining subject consent and the unidentifiable coding of subject information.

### Example 1.4. AD Animal Models

This Example utilized a sample size of five mice (2 males and 3 females) for each of the following groups: APP (Tg2576/C57BL/6J; SJL), humanized Aβ knock-in (hAβ-KI/C57BL/6J), TAU (P301L/FVB/N) transgenic mice, and their respective wildtype counterparts (C57BL/6J, SJL, FVB). The mice were housed under standard laboratory conditions, with an ambient temperature ranging from 22 to 24° C. The mice were subjected to a 12-h light and 12-h dark cycle with the lights being switched on at 6:00 AM and switched off at 6:00 PM, and they were provided ad libitum access to food and water throughout the study. The euthanasia of animals was performed in accordance with the recommendations of the Panel on Euthanasia of the American Veterinary Medical Association, and at the end of each experiment, mice were euthanized by cervical dislocation.

### Example 1.5. Mutant APP cDNA Construct

A mutant APP Swe/IND cDNA clone (pCAX-APP Swe/Ind) was acquired from Addgene (Plasmid #30145, Addgene, Watertown, MA). Subsequently, the genetic material was subcloned into a mammalian expression vector known as pRP-Puro-CAG. The pRP vector is constructed with a pUC backbone, which includes a cytomegalovirus (CMV) promoter and an SV40 polyadenylation site. Additionally, the vector incorporates puromycin selection to facilitate stable transfection. The NCBI sequence hAPP [NM 201414.2]*(K595 N M596 L V642F) was employed to validate the sequence output. To evaluate the upregulation of miR-126969-5p in conditions mimicking AD in a controlled in vitro setting, Applicant conducted a series of experiments utilizing differentiated HT22 cells.

### Example 1.6. MiRNA PC-5P-12969 Expression Vector

The miRNA PC-5P-12969 expression vector (pRP [Exp]-U6>miRNA PC-5P-12969-CAG>EGFP) was purchased from Vector Builder (Cyagen Biosciences, Santa Clara, CA, USA). The expression vector was a 5124 bp plasmid propagated in the Stbl3 host and contained an Enhanced Green Fluorescent Protein (EGFP) encoding gene. The main components of the miRNA PC-5P-12969 construct were 1) the U6 promoter for the Pol III promoter, which drives the expression of small RNAs; 2) Precursor sequence of miRNA PC-5P-12969, which transcribes the mature miRNA PC-5P-12969; 3) Loop sequence that facilitates the folding of shRNA into a hairpin; 4) Terminator sequence that allows the termination of small RNA, transcribed by Pol III RNA polymerase; 5) EGFP, a commonly used green fluorescent for proteins that is ranked high in brightness, photostability, and pH stability among all fluorescent proteins; and 6) pUC origin of the replication that facilitates plasmid replication in E. coli and regulates high-copy plasmid numbers (500-700). The ampicillin encoding gene allows E. coli to be resistant to ampicillin.

Applicant employed two different conditions: 1) miRNA-PC-5P-12969+mAPP condition (Pre-condition: Cells were first treated with the miRNA-PC-5P-12969 overexpression vector for 24 h. Then, the cells were treated with the mAPP vector for another 24 h) and 2) mAPP+miRNA-PC-5P-12969 (Post-condition: Cells were first treated with the mAPP vector for 24 h. Then, the cells were treated with the miRNA-PC-5P-12969 over-expression vector for another 24 h) to understand the better role of miRNA-PC-5P-12969 in AD state.

### Example 1.7. Transfection of Mutant APP cDNA and miRNA PC-5P-12969 Agomirs, Antagomirs, and Vectors

The HT22 cells were cultured in media devoid of antibiotics within 6-well plates one day prior to transfection. On the subsequent day, when the cells had achieved 80% confluence, they underwent transfection with a final concentration of 40 nM of miRNA PC-5P-12969 agomir, miRNA PC-5P-12969 antagomir (Applied Biological Materials, Richmond, BC, Canada), miRNA PC-5P-12969 vector, and mAPP cDNA. To create mutant APP overexpressing-HT22 cells, Applicant transfected HT22 cells with mAPP. This transfection process was carried out using Lipofectamine™ 3000 reagents (Invitrogen Life Tech-nologies, Carlsbad, CA, USA) per the manufacturer's instructions.

### Example 1.8. Cell Viability and Apoptotic Assay

The viability of HT22 cells in both the control and treatment groups (n=4) was evaluated by measuring the reduction of 3-(4,5-dimethyl-thiazol-2-yl)-2,5-diphenyl-tetrazolium bromide (MTT) to formazan, a process that is dependent on mitochondrial activity. The cells were subjected to trypsinization until detachment from the cell culture plate was achieved. Subsequently, they were centrifuged at a force of 300 g for a duration of 3 min. The supernatant was then removed, and the cells were resuspended in 1× phosphate-buffered saline (PBS). A volume of 20 microliters of cells was transferred to the counting chamber in order to assess cell viability.

The cell-based apoptosis assay was conducted in accordance with the methodology previously outlined in Applicant's laboratory. In summary, subsequent to the application of treatment, HT22 cells were collected through the utilization of trypsin, followed by centrifugation at 300 g for a duration of 3 min. The pellets underwent a washing process using a 1×PBS solution. The enumeration of cells was conducted utilizing a hematocytometer, resulting in the collection and subsequent resuspension of 100,000 to 150,000 cells in 40 μl of an Annexin V binding buffer. The binding buffer containing the cells was supplemented with Annexin V-FITC reagent (green) and PI (red), with each reagent being added in a volume of five microliters. The solution was gently mixed through pipetting in an upward and downward motion. The sample was subsequently placed in an incubator for a duration of 15 min at ambient temperature while ensuring the absence of light.

Following the incubation period, a volume of 250 μl of 1×PBS was introduced and subjected to centrifugation at a force of 300 g for a duration of 3 min. The cell pellets were resuspended in 80 μl of an Annexin V binding buffer and subsequently evaluated for the purpose of apoptosis analysis. The Cellometer Vision CBA Image Cytometry System (Nexcelom Bioscience, LLC, Lawrence, MA, USA) was utilized for measuring cell viability and apoptosis.

### Example 1.9. Luciferase Reporter Assay

In order to validate the binding site interaction between miRNA PC-5P-12969 and the 3′-UTR of the GSK3α gene, SH-SY5Y cells were cultured in a 24-well plate at a density of 5×10⁴ cells per well for 24 h prior to transfection. The cells were subsequently transfected with the GSK3α 3′-UTR target expression clone (HmiT008468-MT06) for the human GSK3 (NM 019884.2) and miRNA target clone control vector having mutated [with two sites both mutated (GCTCCTG->GCAGGAC)] 3′-UTR of GSK3α gene pEZX-MT06 (CS-HmiT008468-MT06-01) (Genecopoeia, Rockville, MD, USA).

Cells were also transfected with miRNA PC-5P-12969 agomirs and antagomirs (Applied Biological Materials, Inc., Richmond, BC, Canada). Luciferase activity was assessed 24 h post-transfection using the Luc-Pair™ Duo-Luciferase Assay Kit 2.0 (Genecopoeia, Rockville, MD, USA) following the guidelines provided by the manufacturer. The samples were measured using an illuminometer.

### Example 1.10. MiRNAs Extraction from Tissues and Cell Pellets

The isolation of total RNA was conducted on 80 mg of tissues and cell pellets using the TriZol RT reagent (Ambion, USA), following the guidelines provided by the manufacturer. The extraction of miRNAs and subsequent synthesis of cDNA were conducted. The analysis of RNA quality and quantity was performed using NanoDrop analysis. The absorbance value (A260/A280) for each RNA sample ranged from 1.8 to 2.0. The synthesis of complementary DNA (cDNA) was performed using 1 g of RNA and the miRNA First-Strand cDNA synthesis kit from Agilent Technologies Inc., following the protocol previously described.

### Example 1.11. Real-Time RT-PCR

The real-time quantitative reverse transcription polymerase chain reaction (qRT-PCR) procedure was conducted. In summary, the experimental setup consisted of a reaction mixture comprising 1 μl of a miRNA-specific forward primer (10 μM), 1 μl of a universal reverse primer (3.125 μM) from Agilent Technologies Inc., CA, USA, 10 μl of 2×SYBR Green PCR master mix from Applied Biosystems, NY, USA, and 1 μl of complementary DNA (cDNA). A 20 μl final volume was achieved by adding RNase-free water to the mixture. Primers for miRNA PC-5P-12969 (Forward: 5′-GCAGGAGCCGGGACTGGCTTC-3′; SEQ ID NO: 2), SnoR-202 (5′-AGTACTTTTGAACCCTTTTCCA-3′; SEQ ID NO: 3), and U6snRNA (P1: 5′-CGCTTCGGCAGCACATATAC TAA-3′ (SEQ ID NO: 4) and Reverse: 5′-TATGGAACGCTTCACGAATTTGC-3′ (SEQ ID NO: 5)), GSK3 (Forward: 5′-CCCAGAT CAAAGCTCACCCTT-3′ (SEQ ID NO: 6) and Reverse: 5′-AGAGTGCAATGGCCTCAGGT-3′ (SEQ ID NO: 7)), APP (Forward: 5′-GAGGTGGTCCGAGTTCCCAC-3′ (SEQ ID NO: 8) and Reverse: 5′-AATGGGCATGCTCGTTCTCG-3′ (SEQ ID NO: 9)) were synthesized commercially (Integrated DNA Technologies, Inc., Iowa, USA). In order to standardize the measurement of miRNA expression, the levels of SnoR-202 and U6 snRNA (small nuclear RNA) expression were also assessed in the tissue and cells. These small RNA molecules served as internal controls for mice and humans, respectively.

The reaction mixture for each sample was prepared in triplicate. The experiment was conducted using the QuantStudio™ 5 Real-Time PCR System (Applied Biosystems, USA). The protocol and fold change of miRNA PC-5P-12969 were computed.

### Example 1.12. Immunoblotting Analysis

Applicant performed immunoblotting analysis as previously described. In this Example, protein extracts were acquired from the HT22 cell pellets through a process of homogenization using the RIPA lysis and extraction buffer, following the guidelines provided by the manufacturer (Thermo Scientific, Catalog number: 89901). The protein concentrations were determined using the BCA protein assay method. In each lane, a total of forty micrograms of protein were loaded for the GSK3α and AβPP proteins in both transfected and untransfected cells expressing mAPP and the miRNA PC-5P-12969 vector.

The housing keeping protein, beta-actin, was employed as a loading control to normalize the expression levels of the target proteins, GSK3α and AβPP, in cells that were subjected to treatment with a mAPP and miRNA PC-5P-12969 vector. The samples were subjected to electrophoresis using a 4-20% Mini-PROTEAN® TGX Precast Protein Gels (BIO-RAD). Subsequently, the proteins were transferred to PVDF membranes using the Trans-Blot Turbo Transfer System (BIO-RAD). The membranes were blocked by treating them with a solution of 5% bovine serum albumin (BSA) for a duration of 1 h.

The monoclonal primary antibodies were diluted at a ratio of 1:1000 in a solution containing 2% BSA. These diluted antibodies were then utilized for hybridization over a period of one night at a temperature of 4° C. The hybridization process involved the use of two specific antibodies, namely GSK3 (D75D3, Catalog number: 5676), obtained from Cell Signaling in the Netherlands, and APP (6E10, catalog number: 803001), obtained from Biolegend in San Diego, CA. The membranes underwent a series of washing steps using a TBST buffer, which was repeated three times at 10-min intervals. Subsequently, the membranes were subjected to a 1-h incubation period with secondary antibodies that were suitable for the experiment. This was followed by three additional washing steps at 10-min intervals. The blots were identified through chemiluminescent detection using the Azure 280 chemiluminescent imaging system (Azure Biosystems, Inc, Dublin, CA).

### Example 1.13. Mitochondrial Respiration Analysis

Applicant conducted a mitochondrial respiration analysis using a previously established methodology. In this Example, HT22 cells, which had undergone subculture for a maximum of 16 passages, were seeded in a 6-well plate and allowed to grow overnight. The following day, the cells were transfected with mAPP and miRNA PC-5P-12969 plasmid vectors for a duration of 24 h. Subsequently, HT22 cells were subjected to trypsinization and subsequently cultured in an XFe96 Seahorse plate at a cellular density of 10,000 cells per well. The cells were cultured in 80 μl of DMEM medium in each well, with the exception of four wells (A1, A12, H1, and H12) that were designated for background correction. These wells were blanked with 80 μl of growth medium without cells. Subsequently, the plate was placed within a cell culture hood, allowing it to equilibrate at a temperature range of 20° C. to 25° C. for a duration of 1 h. This procedure ensures the uniform distribution of cells and minimizes any potential edge effects.

The cells were subsequently placed in a cell culture incubator and incubated overnight. On the subsequent day, HT22 cells were subjected to incubation with assay media, specifically XF DMEM medium containing 10 mM glucose, 2 mM glutamine, and 1 mM pyruvate, with a pH of 7.4. This incubation took place in a CO₂-free incubator at a temperature of 37° C. for a duration of 1 h. Following this incubation period, the plate was loaded into the XFe96 analyzer. The measurement of oxygen consumption was conducted utilizing a Seahorse XFe96Analyzer (Agilent Tech-nologies in Santa Clara, CA). The XF Cell Mito Stress Test (Cat No: 103708-100, Seahorse Agilent Technologies) was employed for this purpose. Subsequently, oligomycin (1.5 M), FCCP (1 M), and a combination of rotenone (0.5 M) and antimycin A (0.5 M) were added in a sequential manner to each well at designated time intervals. The methodology for calculating basal respiration and ATP linked was implemented in accordance with the guidelines outlined in the Seahorse Operator's Manual. The experiments were conducted a minimum of three times.

### Example 1.14. Transmission Electron Microscopy

The experiments were conducted using 100-mm Petri dishes. HT22 cells were subjected to transfection using a miRNA PC-5P-12969 vector and a mutant APP cDNA. Additionally, co-transfection was performed using the mAPP and the miRNA PC-5P-12969 vectors (Pre and Post-Condition). Following the incubation period, the cells were rinsed twice with 1×PBS. The cell pellets were dissolved in a fixative solution consisting of 2.5% glutaraldehyde, 1.5% paraformaldehyde, and 0.1 M sodium cacodylate buffer for a duration of 2 h at ambient temperature. The cells were extracted from the fixative solution and transferred into 1 ml of a new fixative solution by scraping. The cells were subjected to incubation at ambient temperature for a duration of 30 min. The cells were subjected to centrifugation at a force of 300 g for a duration of 3 min.

### Example 1.15. Statistical Analysis

Data were represented as mean±standard error of the mean (SEM). The qRT-PCR validation analysis was based on the 2^{-ΔΔCT} value of genes in each sample from AD and control groups. The Cq values of miRNA were transformed and displayed as LnΔCq for statistical analysis. Conclusions were drawn based on statistical analyses conducted using GraphPad™ PRISM software (version 9.3.1; GraphPad Software). Receiver operating characteristic (ROC) curve analysis was employed to assess the sensitivity and specificity of the measured variable as an AD biomarker. The student's t-test was utilized for analyzing two groups of samples, and one-way ANOVA was performed, followed by Tukey's test for multiple comparisons. Group comparisons were considered significant when the p-value was less than 0.05 (p<0.05).

### Example 1.16. MiRNA PC-5P-12969 Expression in AD Postmortem Brains

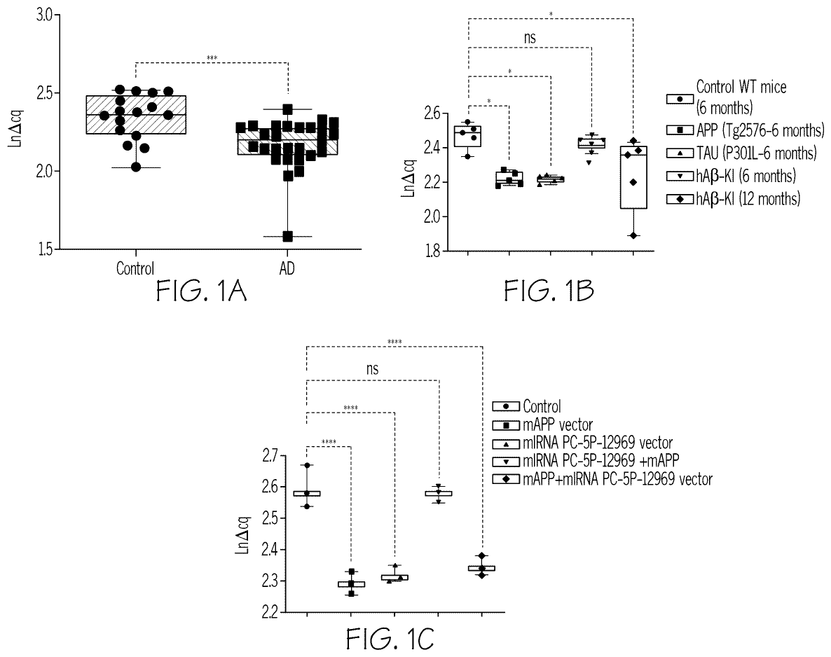

Applicant formulated the hypothesis that examining the molecular response to aging and oxidative stress in the brain at the miRNA level could provide valuable insights into the neuronal reaction. To explore this hypothesis, Applicant conducted an analysis of hsa-miRNA PC-5P-12969 expression in postmortem AD brains (n=27) as well as control brains (n=15) utilizing real-time RT-PCR. Applicant's investigation revealed a noteworthy upregulation of hsa-miRNA PC-5P-12969 (p=0.0008) in the AD brains when compared to the control brains, as depicted in FIG. 1A. The noticeable alteration in the expression of miRNA PC-5P-12969 molecules in human brains between AD cases and control subjects implies that the heightened miRNA PC-5P-12969 could hold relevance for AD concerning potential therapeutic strategies and the progression of the disease.

### Example 1.17. MiRNA PC-5P-12969 Expression in AD Transgenic Mice

miRNA PC-5P-12969 demonstrated promising results in its expression within postmortem AD brains. Furthermore, Applicant investigated the expression of miRNA PC-5P-12969 in cortical tissues obtained from different AD transgenic mice: 6-month-old APP (Tg2576; n=5), TAU (P301 L; n=5), C57BL/6 wild-type mice (n=5), along with 6 and 12 moth-old Aβ knock-in (hAβ-KI; n=5). The findings revealed an elevated expression of mmu-miRNA PC-5P-12969 within the cerebral cortex tissues of both APP mice (p=0.0117) and TAU mice (p=0.0110) in comparison to the wild-type control mice (FIG. 1B). Remarkably, the expression of mmu-miRNA PC-5P-12969 remained unchanged in 6-month-old hAβ-KI mice (p=0.9606), whereas a significant upregulation was observed in 12-month-old hAβ-KI mice (p=0.0362). The substantial elevation in mmu-miRNA PC-5P-12969 expression among the AD transgenic mice lends credence to its potential involvement in Aβ-mediated AD pathogenesis.

### Example 1.18. MiRNA PC-5P-12969 Expression in mAPP Treated HT22 Cells

To evaluate the upregulation of mmu-miRNA-126969-5p in conditions mimicking AD in a controlled in vitro setting, Applicant conducted a series of experiments utilizing differentiated HT22 cells. These cells underwent distinct treatments, namely: 1) untreated HT22 cells, 2) cells treated with the miRNA PC-5P-12969 vector, 3) cells treated with the mAPP plasmid vector, 4) cells treated with a combination of miRNA PC-5P-12969 and mAPP vectors (pre-condition), and 5) cells treated with both mAPP and miRNA PC-5P-12969 vectors (post-condition). Through quantitative qRT-PCR analysis, Applicant observed a significant upregulation of miRNA PC-5P-12969 in cells transfected with the mAPP vector (p<0.0001), the miRNA-PC-5P-12969 vector (p<0.0001), and the post-condition group (p=0.0001). However, no significant alteration was noted in the pre-condition group (p=0.9693) (FIG. 1C).

### Example 1.19. Effects of miRNA PC-5P-12969 and Mutant APP Vectors on Cell Viability and Apoptotic Cell Death

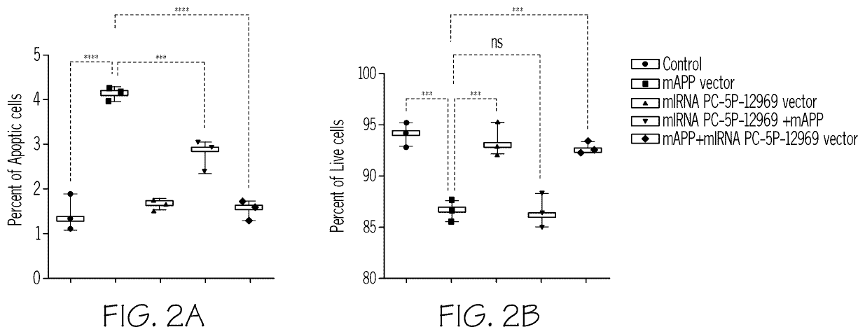

To investigate the impact of mAPP and miRNA PC-5P-12969 on both cell survival and apoptotic cell death, Applicant conducted transfections using mAPP cDNA and miRNA PC-5P-12969 vectors. Notably, a substantial increase in apoptotic cell death was observed in the mAPP-transfected cells in comparison to the untransfected cells (p<0.0001). In contrast, co-transfection involving both mAPP and miRNA PC-5P-12969 (mAPP+miRNA PC-5P-12969) led to a significant reduction in apoptotic cell death (p<0.0001) as compared to cells solely transfected with mAPP (FIG. 2A). Furthermore, cell viability experienced a marked increase (p=0.0003) upon transfection with miRNA PC-5P-12969, when contrasted with mAPP treated cells (FIG. 2B). Correspondingly, in cells co-transfected with the mAPP and miRNA PC-5P-12969 vector, cell viability exceeded that of cells solely transfected with mAPP (p=0.0008). These findings collectively point towards the protective role of miRNA PC-5P-12969 against the toxicity induced by mAPP.

### Example 1.20. MiRNA PC-5P-12969 Interaction with the GSK3α Gene

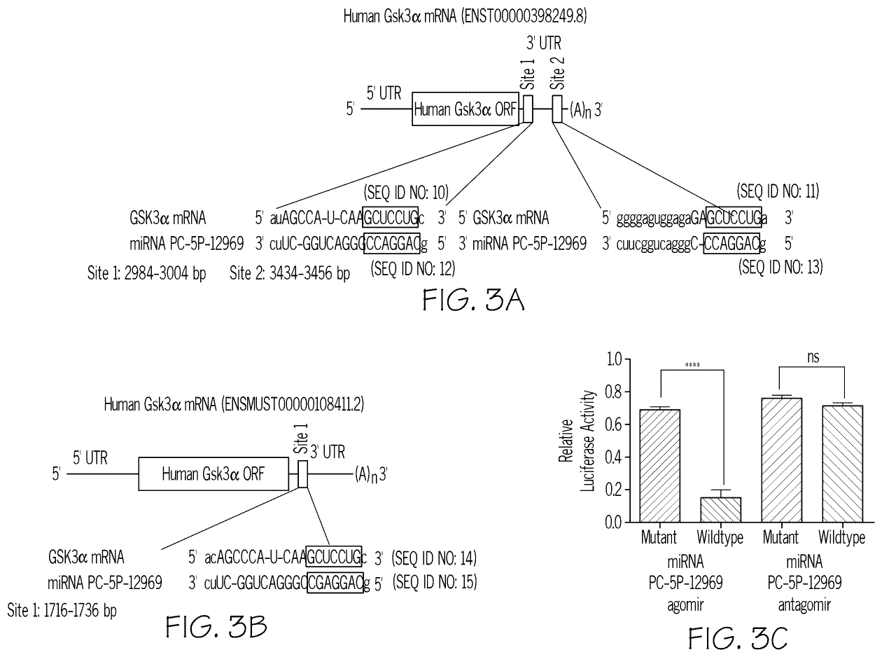

Applicant's recent computational analysis focused on miRNA target prediction, which has unveiled the existence of two distinct binding sites for miRNA PC-5P-12969 within the 3′ untranslated region (UTR) of the human GSK3 gene (ENST00000398249.8). These binding sites are located at sequence positions 2984-3004 and 3434-3456. Furthermore, a single binding site for the miRNA PC-5P-12969 was identified within the 3′-UTR of the mouse GSK3α gene (ENSMUST00000108411.2) at sequence position 1716-1736, as illustrated in FIGS. 3A-3B.

To explore the potential interaction between miRNA PC-5P-12969 and the human GSK3α gene, Applicant conducted luciferase reporter assays. In this experimental setup, luciferase reporter constructs containing both the wild-type (WT) and mutant type (MT) 3′-UTRs of the GSK3α were co-transfected into SH-SY5Y cells along with miRNA PC-5P-12969 agomir and antagomir. The luciferase assays demonstrated a significant decrease (p<0.0001) in the expression of the WT 3′-UTR of the GSK3α when co-transfected with miRNA PC-5P-12969 agomir, compared to their respective controls (FIG. 3C). This outcome strongly suggests the presence of evolutionarily conserved binding sites for miRNA PC-5P-12969 within the 3′-UTR of the GSK3 gene.

To ascertain that this observed effect was not solely attributed to the elevated expression of miRNA PC-5P-12969, Applicant conducted parallel experiments using miRNA PC-5P-12969 antagomir. Remarkably, the luciferase activity remained unaltered, indicating a specific and targeted regulatory influence of miRNA PC-5P-12969 on the 3′-UTR of the GSK3α gene.

### Example 1.21. GSK3α and APP Gene Expression Analysis

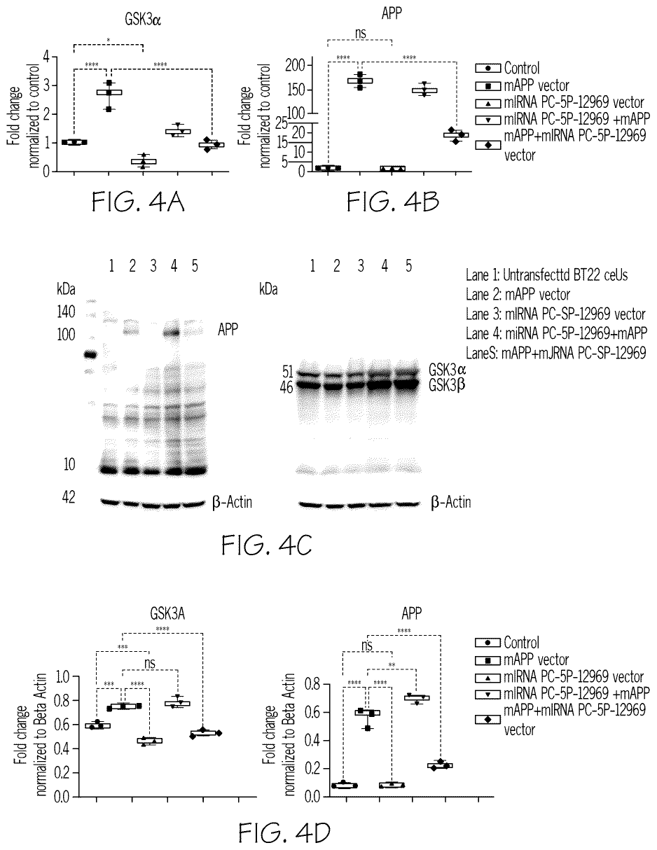

To confirm the regulation of GSK3α and APP gene by miRNA PC-5P-12969, cells were transfected with miRNA PC-5P-12969 vector in mAPP condition (Pre- and Post-condition), followed by the quantification of GSK3α and APP mRNA expression at 24-48 h post-transfection. qRT-PCR analysis showed the significant downregulation of GSK3α (p<0.0001) and APP (p<0.0001) in cells transfected with miRNA PC-5P-12969 vector alone treatment and also in the post-condition compared to the mAPP group, whereas in the mAPP treatment group showed the upregulation of GSK3α and APP gene expression (FIGS. 4A-4B).

### Example 1.22. Effect of miRNA PC-5P-12969 on Protein Levels of GSK3α and AβPP

Applicant proceeded to investigate the expression of miRNA PC-5P-12969 within cells transfected with the miRNA PC-5P-12969 vector, mAPP, as well as pre- and post-condition groups. Furthermore, Applicant examined the impact of miRNA PC-5P-12969 overexpression on the expression of GSK3α and mAPP. Applicant's analysis through immunoblotting corroborates the mRNA data, revealing a noteworthy decrease in the levels of GSK3α (p<0.0001), and full-length AβPP (p<0.0001) within cells transfected with miRNA PC-5P-12969 compared to the mAPP. This reduction was similarly observed in cells co-transfected with the mAPP and miRNA PC-5P-12969 (post-condition), when compared to cells solely expressing mAPP (FIGS. 4C-4D). These findings strongly imply that miRNA PC-5P-12969 governs the modulation of GSK3α and full-length AβPP.

### Example 1.23. MiRNA PC-5P-12969 Enhances Mitochondrial Respiration Rate

To gain a deeper understanding of the underlying mechanisms involving miRNA PC-5P-12969, Applicant investigated its effects on the maximal oxygen consumption rate (OCR) and adenosine triphosphate (ATP) production within mouse hippocampal HT22 cells. This exploration encompassed various treatment conditions, including overexpression vector (OEV) of miRNA PC-5P-12969, mAPP, a combination of OEV miRNA PC-5P-12969 and mAPP (pre-condition), mAPP combined with miRNA PC-5P-12969 (post-condition), along with their corresponding control groups. To facilitate these assessments, Applicant employed an XFe96-well Extracellular Flux Analyzer from Seahorse Bioscience, which enabled the monitoring of mitochondrial metabolism through the quantification of OCRs. The methodology involved evaluating OCRs in both the basal state and subsequent to the introduction of specific agents. Oligomycin was utilized to inhibit ATP synthesis, while FCCP was employed to decouple ATP synthesis from the electron transport chain. Furthermore, the inclusion of rotenone and antimycin A was instrumental in blocking the activity of complex I and III within the electron transport chain, respectively.

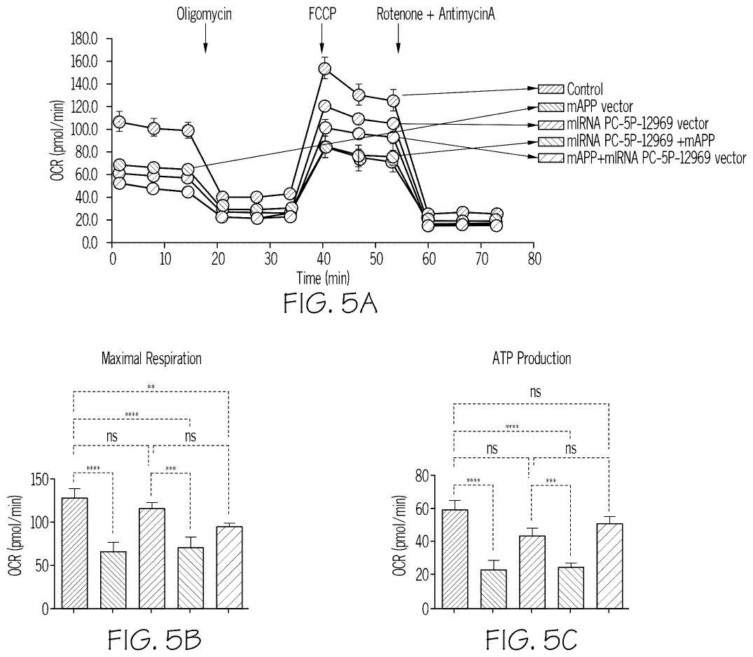

The results of Applicant's Example demonstrated noteworthy findings. The overexpression of miRNA PC-5P-12969, particularly in the post-condition group, exhibited a significant elevation in maximal mitochondrial respiration and ATP production when contrasted with the mAPP group (FIGS. 5A-5C). However, the pre-condition group did not exhibit any discernible impact on maximal respiration and ATP production. Collectively, Applicant's data strongly imply that miRNA PC-5P-12969 plays a pivotal role in augmenting both the rate of mitochondrial respiration and ATP production.

### Example 1.24. Effect of miRNA PC-5P-12969 on Mitochondrial Morphology

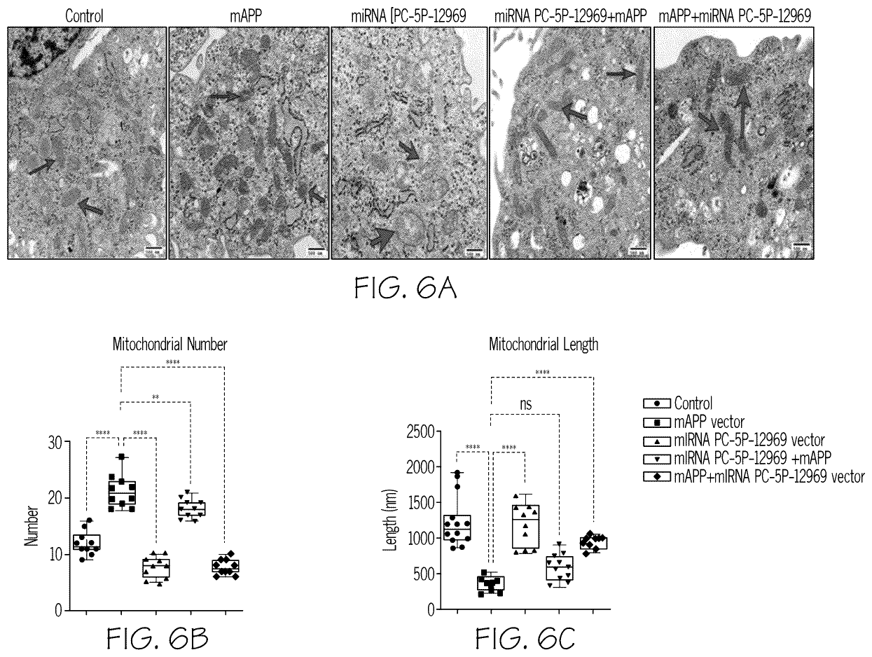

Applicant investigated mitochondrial quantity and length within mAPP treated HT22 cells, following transfection with miRNA PC-5P-12969, aiming to ascertain the impact of miRNA PC-5P-12969 on mitochondrial morphology. The results presented in FIGS. 6A-6C illustrate a notable increase in the count of mitochondria within cells transfected with mAPP cDNA when contrasted with control cells (p<0.0001), suggesting a fragmentary influence of mAPP on mitochondria. In contrast, cells co-transfected with miRNA PC-5P-12969 and mAPP (post-condition) exhibited a significant reduction in mitochondrial count in comparison to those cells transfected with mAPP alone (p<0.0001). Furthermore, an evident decrease in mitochondrial length was observed in mAPP overexpressing-HT22 cells (p<0.0001), while cells co-transfected with miRNA PC-5P-12969 and mAPP displayed an augmentation in mitochondrial length compared to mAPP overexpressing-HT22 cells alone (p<0.0001). These findings collectively underscore the potential of miRNA PC-5P-12969 to regulate and/or rectify the aberrant mitochondrial dynamics induced by the detrimental effects of mAPP.

### Example 1.25. Receiver Operating Characteristics Curve Analysis

In order to assess the diagnostic efficacy of miRNA PC-5P-12969 expression in patients with AD, Applicant generated a receiver operating characteristics (ROC) curve utilizing the (ACT) values of miRNA PC-5P-12969 in both AD patients and individuals without the condition. The analysis revealed a noteworthy Area Under the ROC Curve (AUROC) value for miRNA PC-5P-12969 (AUROC=0.80), accompanied by a 95% confidence interval spanning from 0.6488 to 0.9561 (p=0.0013), when comparing AD brain samples to the healthy control group. This finding underscores the significance of miRNA PC-5P-12969 as a potential biomarker for AD diagnosis.

### Example 1.26. Summary

AD is a profoundly destructive neurodegenerative ailment characterized by a progressive deterioration in cognitive functions, memory loss, and alterations in behavior. Despite extensive investigative efforts, the precise origins of AD continue to elude researchers. In recent times, considerable attention has been directed towards the role of miRNAs in the evolution and advancement of AD. miRNAs, which are diminutive non-coding RNA molecules, play a role in post-transcriptional regulation of gene expression by binding to specific mRNAs.

This Example delves into the nascent insights regarding the participation of miRNAs in the pathogenesis of AD, along with their potential utility as diagnostic biomarkers and promising therapeutic targets. Numerous investigations have demonstrated variations in miRNA expression profiles in brain tissues derived from AD patients, contrasting with those from healthy counterparts. These changes seem to exert influence over diverse cellular processes, encompassing neuronal viability, inflammatory responses, and synaptic plasticity. In the present Example, Applicant address the following aspects: 1) the identification of heightened expression of miRNA PC-5P-12969 in postmortem AD brain samples, transgenic mouse models of AD, and cell lines simulating AD conditions; 2) the role of miRNA PC-5P-12969 as a regulator of GSK3; 3) the capacity of miRNA PC-5P-12969 to alleviate toxicity resulting from mAPP; and 4) the impact of miRNA-PC-5P-12969 overexpression on the gene and protein expression of both GSK3α and AβPP.

Consequently, the discovery of a novel miRNA PC-5P-12969 that concurrently targets GSK3α and AβPP presents potential advantages in abating Aβ accumulation. Without being bound by theory, the observed effects could be part of a more complex interplay of various factors. For instance, cells might adapt to overexpression of miRNA PC-5P-12969, triggering protective responses or alternative signaling pathways that lead to a seemingly positive outcome. The protective effect observed in the overexpression experiments may be a compensatory mechanism rather than a direct consequence of miRNA function. Such a miRNA with even a partial regulatory effect on Aβ could prove instrumental in therapeutic advancements.

miR-132 and miR-134 are implicated in synaptic function and plasticity, with downregulation potentially contributing to synaptic dysfunction seen in AD. Conversely, miR-29α and miR-146α have been associated with neuroinflammation, a hallmark of AD pathology. Such miRNA dysregulation suggests a potential role in disease initiation and progression. Emerging evidence suggests that miRNAs may influence these pathogenic processes.

miR-124, for instance, targets BACE1, a key enzyme involved in Aβ production. Dysregulation of miR-124 could lead to increased Aβ production and deposition. Additionally, miR-132 and miR-134, as mentioned earlier, modulate tau phosphorylation levels, potentially contributing to tau pathology. These findings highlight the intricate relationship between miRNA dysregulation and AD-specific proteinopathies.

Numerous studies have extensively documented altered miRNA expression profiles in mouse models of AD. These miRNAs have exhibited the ability to modulate genes involved in crucial pathways associated with AD pathology. For instance, the genetic removal of miR-132/212 has been linked to the promotion of Aβ production and the formation of amyloid plaques in a mouse model of AD known as triple transgenic AD (3×Tg-AD) mice. Another noteworthy finding involves the manipulation of miR-155, which has shown promising results in mitigating neuroinflammatory responses and enhancing cognitive function in AD-afflicted mice. Furthermore, a distinct investigation has revealed the upregulation of miR-342-5p in various transgenic AD mouse models, including APP/PS1, PS1ΔE9, and PS1-M146V.

In this present Example, Applicant investigated the expression levels of miRNA PC-5P-12969 in various contexts, including postmortem brain samples from individuals with AD, an AD transgenic mouse model, and mutant APP overexpressing-HT22 cells. Remarkably, Applicant's findings reveal an upregulation of miRNA PC-5P-12969 expression in AD postmortem brain samples compared to control brain samples. This consistent upregulation pattern was also evident in diverse AD transgenic mouse models, specifically in 6-month-old APP (Tg2576), TAU (P301L), and 12-month-old hAβ-KI mice. However, intriguingly, no such upregulation was observed in 6-month-old hAβ-KI mice. Furthermore, Applicant generated mutant APP overexpressing-HT22 cells using the mAPP vector and assessed miRNA PC-5P-12969 expression.

Strikingly, Applicant's results demonstrated higher expression levels of miRNA PC-5P-12969 in both mAPP-treated cells and the post-treatment group. This intriguing data compels the Applicant to delve deeper into investigating the potential binding site and mechanistic link between miRNA PC-5P-12969 and AD.

The interplay between miRNAs, cell survival, and apoptotic cell death in AD is intricate and multifaceted. Dysregulated miRNA expression can disrupt the delicate balance within cells, resulting in neuronal dysfunction and demise. For instance, the action of miR-29a has been demonstrated in suppressing Bcl-2 expression, a pivotal anti-apoptotic protein, ultimately triggering heightened neuronal apoptosis. In contrast, miR-21 has been implicated in bolstering cell survival by targeting pro-apoptotic elements, such as PDCD4.

Another miRNA, miR-34a, has been identified as a regulator of SIRT1, a protein crucial for cell survival and stress response. Through its inhibition of SIRT1, miR-34a amplifies apoptotic cell death. The intricate equilibrium between pro-survival and pro-apoptotic miRNAs significantly influences the fate of neurons in AD. In the present Example, a substantial escalation in apoptotic cell death was observed in mAPP-transfected cells compared to their untransfected counterparts. Conversely, co-transfection involving both mAPP and miRNA PC-5P-12969 (Post-condition) led to a noteworthy reduction in apoptotic cell death when contrasted with cells solely transfected with mAPP. Moreover, the introduction of miRNA PC-5P-12969 resulted in a marked augmentation of cell viability. Similarly, cells co-transfected with the mAPP and miRNA PC-5P-12969 vector exhibited higher cell viability than those solely transfected with mAPP. However, no significant alterations were observed in the pre-condition group. This suggests that miRNA-PC-5p-12969 may play a specific role only during the disease state, and not before. In other words, mAPP overexpression alone leads to the upregulation of miRNA PC-5P-12969 expression, subsequently reducing the disease pathology. Collectively, these findings strongly indicate the protective role of miRNA PC-5P-12969 against the toxicity induced by mAPP.

GSK3α has emerged as a central player in AD pathogenesis due to its involvement in tau hyperphosphorylation and AβPP processing. miRNA-mediated regulation of GSK3α presents a potential therapeutic avenue for AD by modulating GSK3α expression to mitigate its detrimental effects on tau phosphorylation and AβPP processing. Applicant's current research findings reveal the existence of evolutionarily conserved binding sites for miRNA PC-5P-12969 within the 3′-UTR of the GSK3α gene.

Several miRNAs have been proven to directly target the 3′-UTR of GSK3α mRNA, leading to translational repression and consequent reduction in GSK3 levels. A notable example is miR-26a, which has demonstrated its ability to target GSK3. This targeting mechanism results in a reduction of tau phosphorylation and even an improvement in cognitive deficits observed in mouse models of AD. Similarly, miR-124 has been identified as a suppressor of GSK3α expression, contributing to a decrease in amyloid beta production and a notable enhancement in cognitive function. These findings underscore the significant potential of targeting the miRNA-GSK3α axis as a promising therapeutic approach for treating AD.

Conversely, certain miRNAs such as miR-216a and miR-135a have been reported to indirectly regulate GSK3. Their impact is achieved by targeting upstream regulators or modulators of the GSK3α signaling pathway. This indirect regulation further emphasizes the intricate network of miRNA-mediated control over GSK3activity.

Numerous studies demonstrated that the upregulation/downregulation of specific miRNAs is correlated with overexpression/reduction of AβPP and BACE1 and that miRNA modulation can affect the AβPP and BACE1 levels, potentially reducing Aβ production. These results underscore the importance of novel treatment approaches that may result from identifying miRNA “signatures” in the AD brain and from manipulating these particular miRNAs, which furthers the significance of miRNAs in AD pathogenesis and emphasizes the relevance of these discoveries. APP mRNA is regulated by multiple miRNAs, including miR-20b, miR-101, miR-153, miR-31, miR-346, miR-106a, and miR-520c. Therefore, miRNA targeting of amyloid metabolism may affect the onset and progression of AD and may constitute an important therapeutic strategy.

Applicant observed a significant downregulation of GSK3α and AβPP in cells that were transfected with the miRNA PC-5P-12969 vector alone, as well as in the post-condition group, when compared to the group expressing mAPP. Conversely, the mAPP treatment group exhibited an upregulation in the gene expression of GSK3α and APP. Furthermore, Applicant assessed the impact of miRNA PC-5P-12969 on the protein levels of GSK3α and AβPP. Notably, the levels of GSK3α and full-length APP were reduced in cells transfected with miRNA PC-5P-12969, both compared to the mAPP group and in cells co-transfected with mutant APP and miRNA PC-5P-12969 (post-condition), in comparison to cells solely expressing mAPP. These findings strongly suggest that miRNA PC-5P-12969 plays a pivotal role in modulating the expression of GSK3α and full-length AβPP.

In the context of mitochondrial respiration, miRNAs have demonstrated the ability to target genes involved in diverse aspects of oxidative phosphorylation (OXPHOS), electron transport chain (ETC) complexes, and mitochondrial dynamics. For instance, miR-181c has been identified as a regulator that downregulates multiple components of the ETC, thereby leading to a disruption in mitochondrial respiration and a decline in energy production.

In contrast, the activity of miR-34a has been associated with an increase in mitochondrial fission, resulting in subsequent synaptic damage observed in AD. Emerging research has identified specific miRNAs whose dysregulation contributes to mitochondrial dysfunction in AD. miR-137 has been found to modulate mitochondrial dynamics and function by targeting multiple genes associated with mitochondrial respiration. Furthermore, the dysregulation of miR-29a has been implicated in mitochondrial impairment relevant to AD, attributed to its targeting of the voltage-dependent anion channel, a pivotal regulator of mitochondrial function.

In this Example, Applicant conducted a comprehensive assessment of mitochondrial respiration using the Sea Horse Bioanalyzer. As anticipated, mAPP-HT22 cells exhibited a reduction in mitochondrial respiration. Conversely, treatment with miRNA PC-5P-12969 resulted in an elevation of mitochondrial respiration, indicated by increased maximal OCR and ATP production in mAPP-HT22 cells. Applicant's findings from transmission electron microscopy unveiled elongated mitochondria and a decrease in fragmented and structurally compromised mitochondria in miRNA PC-5P-12969-treated mAPP-HT22 cells. These observations strongly support the notion that miRNA PC-5P-12969 contributes to the enhancement of mitochondrial quality and overall mitochondrial health.

In summary, miRNA PC-5P-12969 has been shown to be upregulated in postmortem brain samples from individuals with AD, as well as in AD transgenic mouse models and mutant APP overexpressing-HT22 cells. This miRNA PC-5P-12969 plays a crucial role in regulating the expression of GSK3 and the APP gene and protein in an in vitro AD state, ultimately enhancing cell survival and reducing apoptotic cell death. Furthermore, miRNA PC-5P-12969 contributes to improved mitochondrial respiration and mitochondrial quality. It is strongly suggested that miRNA PC-5P-12969 is expressed in neurons, underscoring its potential significance. However, it remains imperative to investigate the specificity of miRNA PC-5P-12969's functions across various cell types, including microglia and astrocytes, in future studies.

Remarkably, Applicant's Example represents the first to elucidate the substantial role of miRNA PC-5P-12969 in the progression and pathogenesis of AD. Applicant's brain tissue samples were exclusively from the frontal cortex region. The intricate interplay between miRNA PC-5P-12969 and GSK3α holds profound implications for Applicant's comprehension of AD pathology. The miRNA PC-5P-12969-GSK3-APP axis emerges as a promising target for therapeutic intervention, offering a novel avenue to mitigate the hyperphosphorylation of tau, accumulation of Aβ, and subsequent cognitive decline.

Based on the aforementioned studies, miRNA based therapies could potentially allow for early intervention, targeting the Alzheimer's disease (AD) before extensive neuronal damage has occurred. Such interventions could lead to better treatment outcomes and slower disease progression.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of treating or preventing a neurological disorder in a subject, wherein the method comprises:
administering to the subject at least one microRNA (miRNA), at least one inhibitor of the at least one miRNA, or combinations thereof,
wherein the at least one miRNA comprises miR PC-5P-12969 (SEQ ID NO: 1), an analog thereof, a derivative thereof, or combinations thereof.

2. The method of claim 1, wherein the administering comprises administering the at least one miRNA.

3. The method of claim 2, wherein the administering comprises administering an expression vector that expresses the at least one miRNA.

4. The method of claim 2, wherein the administering comprises administering the at least one miRNA in isolated form.

5. The method of claim 1, wherein the administering comprises administering at least one inhibitor of the at least one miRNA.

6. The method of claim 5, wherein the at least one inhibitor of the miRNA comprises a reverse complement sequence of SEQ ID NO: 1.

7. The method of claim 1, wherein the neurological disorder is selected from the group consisting of Alzheimer's Disease (AD), Huntington's Disease, Parkinson's Disease, Amyotrophic Lateral Sclerosis (ALS), Multiple Sclerosis (MS), dementia, mild cognitive impairment (MCI), Schizophrenia, or combinations thereof.

8. The method of claim 1, wherein the neurological disorder is Alzheimer's Disease (AD).

9. The method of claim 1, wherein the administering comprises intracranial administration.

10. The method of claim 1, wherein the subject is a human being.

11. The method of claim 1, wherein the subject exhibits symptoms of the neurological disorder.

12. A composition comprising at least one microRNA (miRNA), an expression vector expressing the at least one miRNA, at least one inhibitor of the at least one miRNA, or combinations thereof, wherein the at least one miRNA comprises miR PC-5P-12969 (SEQ ID NO: 1), an analog thereof, a derivative thereof, or combinations thereof.

13. The composition of claim 12, wherein the composition comprises the at least one miRNA.

14. The composition of claim 12, wherein the composition comprises an expression vector expressing the at least one miRNA.

15. The composition of claim 12, wherein the composition comprises at least one inhibitor of the at least one miRNA.

16. The composition of claim 15, wherein the at least one inhibitor of the miRNA comprises a reverse complement sequence of SEQ ID NO: 1.

17. A method of diagnosing a neurological disorder in a subject, said method comprising:
obtaining a biological sample from the subject,
detecting miR PC-5P-12969 (SEQ ID NO: 1) levels in the biological sample, and
correlating overexpressed levels of miR PC-5P-12969 in the biological sample to the presence of the neurological disorder in the subject.

18. The method of claim 17, wherein the biological sample comprises a brain tissue of the subject.

19. The method of claim 18, wherein the brain tissue is obtained from the frontal cortex.

20. The method of claim 17, wherein the detecting occurs by a method selected from the group consisting of a polymerase chain reaction (PCR), a reverse transcriptase (RT) PCR, digital droplet PCR, microarray-based miRNA profiling, next-generation sequencing (NGS), northern blotting, in situ hybridization, or combinations thereof.

21. The method of claim 17, wherein the neurological disorder is selected from the group consisting of Alzheimer's Disease (AD), Huntington's Disease, Parkinson's Disease, Amyotrophic Lateral Sclerosis (ALS), Multiple Sclerosis (MS), dementia, mild cognitive impairment (MCI), Schizophrenia, or combinations thereof.

22. The method of claim 17, wherein the neurological disorder is Alzheimer's Disease (AD).

23. The method of claim 17, further comprising a step of implementing a treatment decision based on the diagnosis.

24. The method of claim 23, wherein treatment decision comprises administering a therapeutic agent to the subject.

25. The method of claim 24, wherein the therapeutic agent comprises an inhibitor of miR PC-5P-12969.


SEQUENCE LISTING (15 sequences)

SEQ ID NO: 1 - RNA, 21 nt, synthetic construct
  source 1..21: mol_type = other RNA
gcaggagccgggactggcttc

SEQ ID NO: 2 - DNA, 21 bp, synthetic construct
  source 1..21: mol_type = other DNA
gcaggagccgggactggcttc

SEQ ID NO: 3 - DNA, 22 bp, synthetic construct
  source 1..22: mol_type = other DNA
agtacttttgaacccttttcca

SEQ ID NO: 4 - DNA, 23 bp, synthetic construct
  source 1..23: mol_type = other DNA
cgcttcggcagcacatatactaa

SEQ ID NO: 5 - DNA, 23 bp, synthetic construct
  source 1..23: mol_type = other DNA
tatggaacgcttcacgaatttgc

SEQ ID NO: 6 - DNA, 21 bp, synthetic construct
  source 1..21: mol_type = other DNA
cccagatcaaagctcaccctt

SEQ ID NO: 7 - DNA, 20 bp, synthetic construct
  source 1..20: mol_type = other DNA
agagtgcaatggcctcaggt

SEQ ID NO: 8 - DNA, 20 bp, synthetic construct
  source 1..20: mol_type = other DNA
gaggtggtccgagttcccac

SEQ ID NO: 9 - DNA, 20 bp, synthetic construct
  source 1..20: mol_type = other DNA
aatgggcatgctcgttctcg

SEQ ID NO: 10 - RNA, 19 nt, synthetic construct
  source 1..19: mol_type = other RNA
atagccatcaagctcctgc

SEQ ID NO: 11 - RNA, 22 nt, synthetic construct
  source 1..22: mol_type = other RNA
ggggagtggagagagctcctga

SEQ ID NO: 12 - RNA, 20 nt, synthetic construct
  source 1..20: mol_type = other RNA
cttcggtcagggccaggacg

SEQ ID NO: 13 - RNA, 21 nt, synthetic construct
  source 1..21: mol_type = other RNA
cttcggtcagggcccaggacg

SEQ ID NO: 14 - RNA, 20 nt, synthetic construct
  source 1..20: mol_type = other RNA
acagcccatcaagctcctgc

SEQ ID NO: 15 - RNA, 21 nt, synthetic construct
  source 1..21: mol_type = other RNA
cttcggtcagggccgaggacg